(19) United States
(12) Patent Application Publication
(10) Pub. No.: US 2003/0233103 A1
(43) Pub. Date: Dec. 18, 2003

(54) System and method for exfoliating human skin

(76) Inventors: Kay Liu, Diamond Bar, CA (US); Craig Miller, Diamond Bar, CA (US)

Correspondence Address: Craig Miller, 23719 Jayhawker, Diamond Bar, CA, 91765, US

(21) Appl. No.: 10/170,494

(22) Filed: Jun. 17, 2002

(51) Int. Cl. A61B 17/50

(52) U.S. Cl. 606/131

(57) ABSTRACT

The present invention provides a method for removing dead epidermis layers, tissue debris and dirt without the harsh abrasive and violent materials that are now known in the art. More Specifically, exfoliating the epidermis layers by using an Ultrasonic Device together with an Exfoliating Fluid.

### FIELD OF THE INVENTION

The present invention relates to and exfoliating system that includes devices for dermatology and more particularly to a hand-held instrument together with an exfoliating fluid that is used against the epidermis layer of human skin to remove epidermis surface layers, as well as to remove tissue debris and dirt from the human skin.

### BACKGROUND OF THE INVENTION

Users of cosmetic and dermatological products are increasingly seeking to remove dead cells and dirt from the skin in and effort to rejuvenate the skin. The attempts of rejuvenating the skin are usually caused by the desire to look younger and often times to prepare the skin for other treatments like skin lightening. There have been many attempts to rejuvenate the skin through Microdermabrasion. On typical prior art Microdermabrasion system is shown in the Shadduck U.S. Pat. No. 6,162,232 in which Shudduck teaches the use of crystalline abrasive media that intermixes in the working end of a hand-held device with a plurality of high velocity gas streams that are used to abrasively scrape the epidermis layers of the skin. These methods of removing layers of the epidermis are well-known in the art of dermatology. In general, Microdermabrasion, or scraping the epidermis with an abrasive source is a well-known method of removing unwanted epidermis layers, dirt and tissue debris. In general, the use of a sand or abrasive material together with an air pressure system, where the air-pressure system is used to drive the sand or abrasive material at the epidermis layer of human skin at a high rate of speed to remove epidermis surface layers, as well as to remove tissue debris and dirt from the human skin. Unfortunately, this well-known system of Microdermabrasion is a very violent procedure where the high velocity of sand or abrasive materials tends to over irritate the skin leaving more damage and often penetrating the epidermis and driving abrasive particles into the dermis layer of the skin which leaves the skin subject to dirt and infection.

Other known uses in the art consist of highly reactive chemicals that are placed on the epidermis with an adhesive material where the reactive chemical bonds with the epidermis and is removed with the adhesive material. Unfortunately, these commonly known practices as may affectively remove epidermis layers of the skin they also remove live dermis layers and cause great irritations due to the violent manner at which they remove dead epidermis layers, tissue debris and dirt.

### SUMMARY OF THE INVENTION

The present invention provides a method for removing dead epidermis layers, tissue debris and dirt without the harsh abrasive and violent materials that are well-known in the art.

More specifically, an advantage of the present invention is to provide a method of utilizing a hand-held ultrasonic device together with an exfoliating fluid to remove unwanted epidermis surface layers, as well as to remove tissue debris and dirt without irritating the skin. As a human passes the age of thirty, the renewal rate begins to slow significantly, increasing to about 28 days to complete a full cycle. This results in thicker, duller-looking skin. During this time, glands become less active, making the skin slightly drier. While no skin care product will stop this natural process, the present invention can help the skin rejuvenate by accelerating its renewal process. More Specifically, an ultrasonic device is used together with an exfoliating fluid to remove epidermis surface layers, as well as to remove tissue debris and dirt from the skin. The ultrasonic devise that is used in this invention must operate and produce a low ultrasonic wave that oscillates at a frequency of 10 to 100 kilohertz. Due to the fragility of human skin an ultrasonic device that is used at a high frequency would cause great irritation and possibly permanently damage the skin. When a low frequency ultrasonic device is deposed near the surface of the skin and an ultrasonic wave is generated between the frequencies of 10 to 100 kilohertz. The ultrasonic waves resonate through the epidermis layers of the skin. Unfortunately, a low frequency Ultrasonic device cannot produce enough energy or heat to substantially exfoliate the epidermis layer of the skin. Thus, an additional advantage to the present invention is the use of an exfoliating fluid that assists in the resonation and transportation of the energy and heat produced by the ultrasonic waves while also providing healing minerals to help minimize skin irritation.

### BRIEF DESCRIPTION OF THE DRAWINGS

FIG. 1 illustrates the layers of the skin

FIG. 2 illustrates the exfoliating fluid

FIG. 3 illustrates the present invention in use

### DETAILED DESCRIPTION OF THE INVENTION

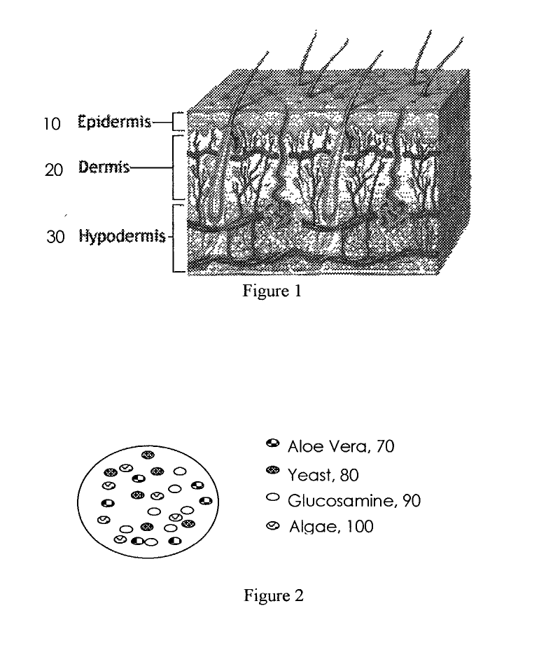

As shown in FIG. 1 there are three basic layers of the human skin, which is shown as the Epidermis, 10, Dermis, 20 and Hypodermis, 30. As the human ages the oil producing glands in the Hypodermis, 30 become less active thus slowing the regeneration process performed by the Dermis, 20 layer of the skin. While the Hypodermis, 30 produces less oil thus causing the Dermis, 20 to become less active at creating new cells to replace the Epidermis, 10.

In the representative embodiment of the exfoliation fluid as shown in FIG. 2, there are four active ingredients used, Aloe Vera, 70, Yeast, 80, Glucosamine, 90, and Algae, 100. The Glucosamine and Yeast are used to assist in the transfer of energy and heat created by the ultrasonic waves from the Ultrasonic Device to the Epidermis. Furthermore, the Algae, 100 and the Aloe Vera, 70 are used to calm and cool the newly exposed Epidermis.

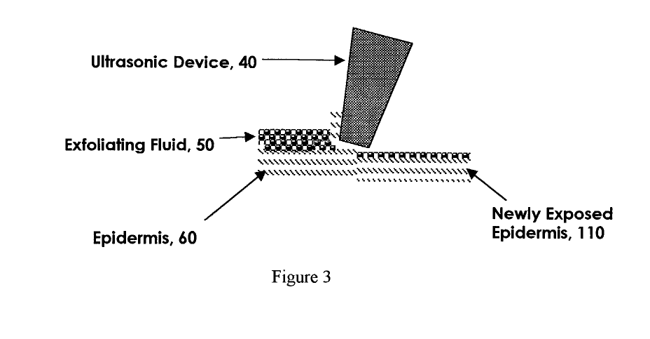

In the representative embodiment of the Exfoliating System as shown in FIG. 3, an Exfoliating Fluid, 50 is placed in contact with the Epidermis, 60. Furthermore an Ultrasonic Device, 40 is deposed adjacent to the Epidermis, 60 and in contact with the Exfoliating Fluid, 50 where the Ultrasonic Device, 40 is enabled to move horizontally across the Epidermis, 60 where Exfoliating Fluid, 50 is present. The method of moving an Ultrasonic Device, 40 horizontally across the Epidermis, 60 where Exfoliating Fluid, 50 is present results in the removal dead epidermis layers, tissue debris and dirt, thus exposing Newly Exposed Epidermis 110.

What is claimed is:

1. An exfoliating system for human skin comprising:
an ultrasonic device capable of producing ultrasonic waves with a frequency at a magnitude between 10 kilohertz to 100 kilohertz and capable of being deposed adjacent to the Epidermis layer of the skin; and
an exfoliating fluid wherein the exfoliating fluid is capable of contacting the epidermis.

2. The exfoliating system of claim 1, wherein the exfoliating fluid is comprised of Aloe Vera, Yeast, Glucosamine, and Algae.

3. The exfoliating system of claim 1, wherein the Exfoliating Fluid is comprised of Aloe Vera, Yeast, Glucosamine.

4. An exfoliating system that exfoliates the Epidermis of human skin comprising the steps of:
placing an exfoliating fluid in contact with the Epidermis, and placing an ultrasonic device disposed adjacent to the epidermis where the ultrasonic device contacts the exfoliating fluid.

5. The exfoliating system of claim 4, wherein the exfoliating fluid is comprised of Aloe Vera, Yeast, Glucosamine, and Algae.

6. A method of exfoliating the epidermis comprising the steps of
applying an exfoliating liquid formed of Aloe Vera, Yeast, Glucosamine, and Algae to the Epidermis so that the exfoliating system contacts the epidermis; and
deposing an ultrasonic device adjacent to the Epidermis whereby contacting the exfoliating liquid; and
moving the ultrasonic device horizontally thereby causing the epidermis to exfoliate.

7. The method of exfoliating the epidermis of claim 6,
wherein the Exfoliating Liquid comprises of Aloe Vera, Yeast, Glucosamine, and Algae.
